SEQUENCE LISTING (24 sequences)

SEQ ID NO: 1 - protein, 609 aa, Homo sapiens [sequence omitted]

SEQ ID NO: 2 - protein, 572 aa, Homo sapiens
MAAPRPSPAISVSVSAPAFYAPQKKFGPVVAPKPKVNPFRPGDSEPPPAPGAQRAQMGRVGEIPPPPPEDFPLPPPPLAGDGDDAEGALGGAFPPPPPPIEESFPPAPLEEEIFPSPPPPPEEEGGPEAPIPPPPQPREKVSSIDLEIDSLSSLLDDMTKNDPFKARVSSGYVPPPVATPFSSKSSTKPAAGGTAPLPPWKSPSSSQPLPQVPAPAQSQTQFHVQPQPQPKPQVQLHVQSQTQPVSLANTQPRGPPASSPAPAPKFSPVTPKFTPVASKFSPGAPGGSGSQPNQKLGHPEALSAGTGSPQPPSFTYAQQREKPRVQEKQHPVPPPAQNQNQVRSPGAPGPLTLKEVEELEQLTQQLMQDMEHPQRQNVAVNELCGRCHQPLARAQPAVRALGQLFHIACFTCHQCAQQLQGQQFYSLEGAPYCEGCYTDTLEKCNTCGEPITDRMLRATGKAYHPHCFTCVVCARPLEGTSFIVDQANRPHCVPDYHKQYAPRCSVCSEPIMPEPGRDETVRVVALDKNFHMKCYKCEDCGKPLSIEADDNGCFPLDGHVLCRKCHTARAQT

SEQ ID NO: 3 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 4 - protein, 223 aa, Homo sapiens [sequence omitted]

SEQ ID NO: 5 - protein, 793 aa, Homo sapiens
MDDFERRRELRRQKREEMRLEAERIAYQRNDDDEEEAARERRRRARQERLRQKQEEESLGQVTDQVEVNAQNSVPDEEAKTTTTNTQVEGDDEAAFLERLARREERRQKRLQEALERQKEFDPTITDASLSLPSRRMQNDTAENETTEKEEKSESRQERYEIEETETVTKSYQKNDWRDAEENKKEDKEKEEEEEEKPKRGSIGENQVEVMVEEKTTESQEETVVMSLKNGQISSEEPKQEEEREQGSDEISHHEKMEEEDKERAEAERARLEAEERERIKAEQDKKIADERARIEAEEKAAAQERERREAEERERMREEEKRAAEERQRIKEEEKRAAEERQRIKEEEKRAAEERQRIKEEEKRAAEERQRARAEEEEKAKVEEQKRNKQLEEKKRAMQETKIKGEKVEQKIEGKWVNEKKAQEDKLQTAVLKKQGEEKGTKVQAKREKLQEDKPTFKKEEIKDEKIKKDKEPKEEVKSFMDRKKGFTEVKSQNGEFMTHKLKHTENTFSRPGGRASVDTKEAEGAPQVEAGKRLEELRRRRGETESEEFEKLKQKQQEAALELEELKKKREERRKVLEEEEQRRKQEEADRKLREEEEKRRLKEEIERRRAEAAEKRQKMPEDGLSDDKKPFKCFTPKGSSLKIEERAEFLNKSVQKSSGVKSTHQAAIVSKIDSRLEQYTSAIEGTKSAKPTKPAASDLPVPAEGVRNIKSMWEKGNVFSSPTAAGTPNKETAGLKVGVSSRINEWLTKTPDGNKSPAPKPSDLRPGDVSSKRNLWEKQSVDKVTSPTKV

SEQ ID NO: 6 - protein, 240 aa, Homo sapiens
MDPRKVNELRAFVKMCKKDPSILHTQEMRFLREWVESMGGTATQKAKSEENTKEEKPDSKVEEDLKADEPSSEESDLEIDKEGVIEPDTDAPQEMGDENAEITEEVMDQANDKKVAAIEALNDGELQKAIDLFTDAIKLNPRLAILYAKRASVFVKLQKPNAAIRDCDRAIEINPDSAQPYKRRGKAHRLLGHWEEAAHDLALACKFDYDEDASAMLKEVQPRAQKIAEHQRKYERKREE

SEQ ID NO: 7 - protein, 710 aa, Homo sapiens
MSDYSTGGPPPGPPPPAGGGGGAGGAGGGPPPGPPGAGDRGGGGPCGGGPGGGSAGGPSQPPGGGGPGIRKDAFADAVQRARQIAAKIGGDAATTVNNSTPDFGFGGQKRQLEDGDQPESKKLASQGDSISSQLGPIHPPPRTSMTEEYRVPDGMVGLIIGRGGEQINKIQQDSGCKVQISPDSGGLPERSVSLTGAPESVQKAKMMLDDIVSRGRGGPPGQFHDNANGGQNGTVQEIMIPAGKAGLVIGKGGETIKQLQERAGVKMILIQDGSQNTNVDKPLRIIGDPYKVQQACEMVMDILRERDQGGFGDRNEYGSRIGGGIDVPVPRHSVGVVIGRSGEMIKKIQNDAGVRIQFKQDDGTGPEKIAHIMGPPDRCEHAARIINDLLQSLRSGPPGPPGGPGMPPGGRGRGRGQGNWGPPGGEMTFSIPTHKCGLVIGRGGENVKAINQQTGAFVEISRQLPPNGDPNFKLFIIRGSPQQIDHAKQLIEEKIEGPLCPVGPGPGGPGPAGPMGPFNPGPFNQGPPGAPPHAGGPPPHQYPPQGWGNTYPQWQPPAPHDPSKAAAAAADPNAAWAAYYSHYYQQPPGPVPGPAPAPAAPPAQGEPPQPPPTGQSDYTKAWEEYYKKIGQQPQQPGAPPQQDYTKAWEEYYKKQAQVATGGGPGAPPGSQPDYSAAWAEYYRQQAAYYGQTPVPGPQPPPTQQGQQQAQ

SEQ ID NO: 8 - protein, 375 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 9 - protein, 434 aa, Homo sapiens
MSIEKIWAREILDSRGNPTVEVDLYTAKGLFRAAVPSGASTGIYEALELRDGDKQRYLGKGVLKAVDHINSTIAPALISSGLSVVEQEKLDNLMLELDGTENKSKFGANAILGVSLAVCKAGAAERELPLYRHIAQLAGNSDLILPVPAFNVINGGSHAGNKLAMQEFMILPVGAESFRDAMRLGAEVYHTLKGVIKDKYGKDATNVGDEGGFAPNILENSEALELVKEAIDKAGYTEKIVIGMDVAASEFYRDGKYDLDFKSPTDPSRYITGDQLGALYQDFVRDYPVVSIEDPFDQDDWAAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGDEARFAGHNFRNPSVL

SEQ ID NO: 10 - protein, 505 aa, Homo sapiens
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 11 - protein, 470 aa, Homo sapiens
MSQAYSSSQRVSSYRRTFGGAPGFPLGSPLSSPVFPRAGFGSKGSSSSVTSRVYQVSRTSGGAGGLGSLRASRLGTTRTPSSYGAGELLDFSLADAVNQEFLTTRTNEKVELQELNDRFANYIEKVRFLEQQNAALAAEVNRLKGREPTRVAELYEEELRELRRQVEVLTNQRARVDVERDNLLDDLQRLKAKLQEEIQLKEEAENNLAAFRADVDAATLARIDLERRIESLNEEIAFLKKVHEEEIRELQAQLQEQQVQVEMDMSKPDLTAALRDIRAQYETIAAKNISEAEEWYKSKVSDLTQAANKNNDALRQAKQEMMEYRHQIQSYTCEIDALKGTNDSLMRQMRELEDRFASEASGYQDNIARLEEEIRHLKDEMARHLREYQDLLNVKMALDVEIATYRKLLEGEESRINLPIQTYSALNFRETSPEQRGSEVHTKKTVMIKTIETRDGEVVSEATQQQHEVL

SEQ ID NO: 12 - protein, 470 aa, Homo sapiens
MSHHPSGLRAGFSSTSYRRTFGPPPSLSPGAFSYSSSSRFSSSRLLGSASPSSSVRLGSFRSPRAGAGALLRLPSERLDFSMAEALNQEFLATRSNEKQELQELNDRFANFIEKVRFLEQQNAALRGELSQARGQEPARADQLCQQELRELRRELELLGRERDRVQVERDGLAEDLAALKQRLEEETRKREDAEHNLVLFRKDVDDATLSRLELERKIESLMDEIEFLKKLHEEELRDLQVSVESQQVQQVEVEATVKPELTAALRDIRAQYESIAAKNLQEAEEWYKSKYADLSDAANRNHEALRQAKQEMNESRRQIQSLTCEVDGLRGTNEALLRQLRELEEQFALEAGGYQAGAARLEEELRQLKEEMARHLREYQELLNVKMALDIEIATYRKLLEGEESRISVPVHSFASLNIKTTVPEVEPPQDSHSRKTVLIKTIETRNGEVVTESQKEQRSELDKSSAHSY

SEQ ID NO: 13 - protein, 449 aa, Homo sapiens
MMLGTEGGEGFVVKVRGLPWSCSADEVQRFFSDCKIQNGAQGIRFIYTREGRPSGEAFVELESEDEVKLALKKDRETMGHRYVEVFKSNNVEMDWVLKHTGPNSPDTANDGFVRLRGLPFGCSKEEIVQFFSGLEIVPNGITLPVDFQGRSTGEAFVQFASQEIAEKALKKHKERIGHRYIEIFKSSRAEVRTHYDPPRKLMAMQRPGPYDRPGAGRGYNSIGRGAGFERMRRGAYGGGYGGYDDYNGYNDGYGFGSDRFGRDLNYCFSGMSDHRYGDGGSTFQSTTGHCVHMRGLPYRATENDIYNFFSPLNPVRVHIEIGPDGRVTGEADVEFATHEDAVAAMSKDKANMQHRYVELFLNSTAGASGGAYEHRYVELFLNSTAGASGGAYGSQMMGGMGLSNQSSYGGPASQQLSGGYGGGYGGQSSMSGYDQVLQENSSDFQSNIA

SEQ ID NO: 14 - protein, 543 aa, Homo sapiens
MEQVNELKEKGNKALSVGNIDDALQCYSEAIKLDPHNHVLYSNRSAAYAKKGDYQKAYEDGCKTVDLKPDWGKGYSRKAAALEFLNRFEEAKRTYEEGLKHEANNPQLKEGLQNMEARLAERKFMNPFNMPNLYQKLESDPRTRTLLSDPTYRELIEQLRNKPSDLGTKLQDPRIMTTLSVLLGVDLGSMDEEEEIATPPPPPPPKKETKPEPMEEDLPENKKQALKEKELGNDAYKKKDFDTALKHYDKAKELDPTNMTYITNQAAVYFEKGDYNKCRELCEKAIEVGRENREDYRQIAKAYARIGNSYFKEEKYKDAIHFYNKSLAEHRTPDVLKKCQQAEKILKEQERLAYINPDLALEEKNKGNECFQKGDYPQAMKHYTEAIKRNPKDAKLYSNRAACYTKLLEFQLALKDCEECIQLEPTFIKGYTRKAAALEAMKDYTKAMDVYQKALDLDSSCKEAADGYQRCMMAQYNRHDSPEDVKRRAMADPEVQQIMSDPAMRLILEQMQKDPQALSEHLKNPVIAQKIQKLMDVGLIAIR

SEQ ID NO: 15 - protein, 249 aa, Homo sapiens
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 16 - protein, 224 aa, Homo sapiens
MPGGLLLGDVAPNFEANTTVGRIRFHDFLGDSWGILFSHPRDFTPVCTTELGRAAKLAPEFAKRNVKLIALSIDSVEDHLAWSKDINAYNCEEPTEKLPFPIIDDRNRELAILLGMLDPAEKDEKGMPVTARVVFVFGPDKKLKLSILYPATTGRNFDEILRVVISLQLTAEKRVATPVDWKDGDSVMVLPTIPEEEAKKLFPKGVFTKELPSGKKYLRYTPQP

SEQ ID NO: 17 - protein, 331 aa, Homo sapiens
MARGGRGRRLGLALGLLLALVLAPRVLRAKPTVRKERVVRPDSELGERPPEDNQSFQYDHEAFLGKEDSKTFDQLTPDESKERLGKIVDRIDNDGDGFVTTEELKTWIKRVQKRYIFDNVAKVWKDYDRDKDDKISWEEYKQATYGYYLGNPAEFHDSSDHHTFKKMLPRDERRFKAADLNGDLTATREEFTAFLHPEEFEHMKEIVVLETLEDIDKNGDGFVDQDEYIADMFSHEENGPEPDWVLSEREQFNEFRDLNKDGKLDKDEIRHWILPQDYDHAQAEARHLVYESDKNKDEKLTKEEILENWNMFVGSQATNYGEDLTKNHDEL

SEQ ID NO: 18 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 19 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 20 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 21 - protein, 198 aa, Homo sapiens
MASGNARIGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 22 - protein, 256 aa, Homo sapiens
MAAAVGRLLRASVARHVSAIPWGISATAALRPAACGRTSLTNLLCSGSSQAKLFSTSSSCHAPAVTQHAPYFKGTAVVNGEFKDLSLDDFKGKYLVLFFYPLDFTFVCPTEIVAFSDKANEFHDVNCEVVAVSVDSHFSHLAWINTPRKNGGLGHMNIALLSDLTKQISRDYGVLLEGSGLALRGLFIIDPNGVIKHLSVNDLPVGRSVEETLRLVKAFQYVETHGEVCPANWTPDSPTIKPSPAASKEYFQKVNQ

SEQ ID NO: 23 - protein, 201 aa, Homo sapiens
MAAAKDTHEDHDTSTENTDESNHDPQFEPIVSLPEQEIKTLEEDEEELFKMRAKLFRFASENDLPEWKERGTGDVKLLKHKEKGAIRLLMRRDKTLKICANHYITPMMELKPNAGSDRAWVWNTHADFADECPKPELLAIRFLNAENAQKFKTKFEECRKEIEEREKKAGSGKNDHAEKVAEKLEALSVKEETKEDAEEKQ

SEQ ID NO: 24 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

(19) United States
(12) Patent Application Publication
(10) Pub. No.: US 2011/0311995 A1
(43) Pub. Date: Dec. 22, 2011

(54) METHOD FOR THE DIAGNOSIS OF SYSTEMIC SCLERODERMA OR OF PULMONARY ARTERIAL HYPERTENSION

(76) Inventors: Luc Mouthon, Saint Mande (FR); Marc Humbert, Issy Les Moulineaux (FR); Cynthia Calzas, Montfermeil (FR); Luc Camoin, Paris (FR); Younes Sahbatou, Saint-Denis (FR)

(21) Appl. No.: 12/998,080

(22) PCT Filed: Sep. 17, 2009
(86) PCT No.: PCT/FR2009/051753

§ 371 Date: Aug. 30, 2011

(30) Foreign Application Priority Data
Sep. 17, 2008 (FR) 0856255

(51) Int. Cl. G01N 33/566

(52) U.S. Cl. 435/7.92; 436/501

(57) ABSTRACT

The invention relates to an in vitro method for detecting systemic scleroderma (SSc) and/or pulmonary arterial hypertension (PAH), or a risk of developing SSc or PAH, which comprises determining the presence and/or the amount of antibodies in a biological sample originating from a patient.

The invention relates to an in vitro method for detecting systemic scleroderma (SSc) and/or pulmonary arterial hypertension (PAH), or a risk of developing SSc or PAH, which comprises determining the presence and/or the amount of antibodies in a biological sample originating from a patient.

### PRIOR ART

Systemic scleroderma (SSc) is a rare disease which is characterized by the occurrence of fibrosis lesions involving the skin and certain viscera such as the lungs, the digestive tract and the heart, and also vascular hyperreactivity responsible for Raynaud's phenomenon and serious manifestations such as renal crisis and pulmonary arterial hypertension (PAH). The physiology of SSc is complex and partially understood. The occurrence of SSc is the result of the disfunctioning of three cell types, B and T lymphocytes responsible for immune disregulation, endothelial cells responsible for vascular abnormalities and fibroblasts responsible for fibrosis lesions.

Ninety percent of scleroderma patients have antinuclear antibodies (ANAs) in their serum. Some autoantibodies are very specific for SSc and mutually exclusive, such as anti-topoisomerase I antibodies (anti-SCI-70) (ATAs) (Tamby et al., 2007), more commonly present in the diffuse forms of the disease, anti-centromere antibodies (ACAs), as a rule associated with the limited cutaneous forms (Moroi et al., 1980), or anti-RNA polymerase III antibodies associated with the diffuse cutaneous forms and with the occurrence of renal crisis (Bunn et al., 1998). ANAs do not have a demonstrated pathogenic role during SSc, but their detection constitutes an aid to early diagnosis of SSc. Other autoantibodies which are not specific for SSc, such as anti-ribonucleoprotein, anti-SSA and anti-SSB antibodies, anti-cardiolipin antibodies or rheumatoid factor are sometimes found during SSc. Anti-endothelial cell antibodies (AECAs) can be detected in 28% to 54% of scleroderma patients. These autoantibodies are capable of inducing the expression of adhesion molecules and of causing endothelial cell apoptosis in the presence of natural killer cells (Bordron et al., 1998). The targets of AECAs during SSc are currently poorly understood and, to date, no endothelial-cell-specific antigen has been identified. On the other hand, DNA topoisomerase 1 (Garcia de la Pena-Lefebvre et al., 2004) and centromeric protein B (Servettaz et al., 2006) have been identified as targets of AECAs in scleroderma patients.

Anti-fibroblast antibodies (AFAs) have been identified in scleroderma patients. These antibodies are capable of activating fibroblasts, of increasing the expression of adhesion molecules such as intercellular adhesion molecule-1 (ICAM) and pro-inflammatory molecules (increase in IL-1α, IL-1β and IL-6 mRNA levels) and also collagen synthesis (Chizzolini et al., 2002). It has recently been demonstrated that AFAs can bind to topoisomerase 1 adsorbed at the surface of fibroblasts (Henault et al., 2006). AFAs have been found, by ELISA (Enzyme-linked immunosorbent assay), in 30% of patients presenting PAH associated with SSc (Tamby et al., 2006). Pulmonary arterial hypertension (PAH) is a rare pathological condition responsible for the occurrence of right cardiac decompensation which can result in death. PAH diagnosis is established by right catheterization, which makes it possible to measure average pulmonary arterial pressure of greater than or equal to 25 mmHg while resting, in the absence of elevated pulmonary capillary pressure (Rubin, 1997). The occurrence of PAH is the result of a chronic obstruction of the small pulmonary arteries secondary to the proliferation of endothelial cells, vascular smooth muscle cells and fibroblasts (Dorfmuller et al, 2003). In particular, neomuscularization of the small peripheral pulmonary arteries, which are normally nonmuscularized, is a characteristic common to all forms of remodeling associated with PAH. PAH may be idiopathic, i.e. sporadic, but also familial, associated with the taking of anorexigenics (dexfenfluramine), or associated with a certain number of pathological conditions including infection with human immunodeficiency virus (HIV). PAH can also be associated with collagenosis, such as SSc (Hachulla et al, 2005), Sharp's syndrome (or mixed connective tissue disease), or more rarely systemic lupus erythematosis. Approximately 8% to 12% of scleroderma patients develop PAH, responsible for a high mortality. In addition, during idiopathic PAH, marks of autoimmunity, namely antinuclear antibodies or anti-thyroglobulin antibodies, are from time to time found.

The presence of anti-endothelial cell antibodies (Tamby et al, 2005) and of anti-fibroblast antibodies (Tamby et al, 2006) has been reported during idiopathic PAH or SSc-associated PAH. However, the predictive value of these antibodies in the occurrence of PAH has not been studied and the potential role of autoimmune phenomena in the pathogenesis of idiopathic PAH remains uncertain (Mouthon et al, 2005).

In most cases, PAH is screened for when the patient presents stage III or IV dyspnea. When the patient is monitored for a chronic disease such as SSc, PAH is screened for by annual echocardiography.

A simple and reliable test to screen for SSc and/or PAH is still lacking, and would be invaluable for the earliest possible diagnosis, which would make it possible to rapidly set up therapeutic strategies for improving the condition of the patient and the survival chances of said patient. The subject-matter of the invention is such a test.

### SUMMARY OF THE INVENTION

The invention now provides an in vitro method for detecting systemic scleroderma (SSc) and/or pulmonary arterial hypertension (PAH) in an individual, or a risk of developing SSc and/or PAH, which comprises determining the presence and/or the amount of at least one antibody selected from the group consisting of the following antibodies: anti-serum albumin precursor, anti-zyxin, anti-galectin-1, anti-ubiquitin carboxyl-terminal hydrolase isozyme L1, anti-caldesmon, anti-FAM10A4 protein, anti-Far-upstream element-binding protein 2, anti-cytoplasmic actin 2, anti-γ-enolase, anti-protein disulfide-isomerase A3 precursor, anti-desmin, anti-peripherin, anti-heterogeneous nuclear ribonucleoprotein H, anti-stress-induced phosphoprotein 1, anti-triosephosphate isomerase, anti-peroxiredoxin-6, anti-reticulocalbin-1, anti-peroxiredoxin-2, anti-thioredoxin-dependent peroxide reductase mitochondrial precursor, anti-Ran-specific GTPase-activation protein, anti-high mobility group protein B1, anti-tubulin beta-chain and anti-polymerase I and transcript release factor, in a biological sample originating from a patient, the presence of said at least one antibody being an indicator of SSc and/or of PAH, or of a risk of developing SSc and/or PAH.

Preferably, the presence of said at least one antibody in the biological sample is compared with a control value, the presence of said at least one antibody in an amount greater than the control value being an indicator of SSc and/or of PAH, or of a risk of developing SSc or PAH.

Another aspect of the invention is an in vitro method for the prognosis or the monitoring of SSc and/or PAH, which comprises determining the presence and/or the amount of at least one antibody selected from the group consisting of the following antibodies: anti-serum albumin precursor, anti-zyxin, anti-galectin-1, anti-ubiquitin carboxyl-terminal hydrolase isozyme L1, anti-caldesmon, anti-FAM10A4 protein, anti-Far-upstream element-binding protein 2, anti-cytoplasmic actin 2, anti-γ-enolase, anti-protein disulfide-isomerase A3 precursor, anti-desmin, anti-peripherin, anti-heterogeneous nuclear ribonucleoprotein H, anti-stress-induced phosphoprotein 1, anti-triosephosphate isomerase, anti-peroxiredoxin-6, anti-reticulocalbin-1, anti-peroxiredoxin-2, anti-thioredoxin-dependent peroxide reductase mitochondrial precursor, anti-Ran-specific GTPase-activating protein, anti-high mobility group protein B1, anti-tubulin beta-chain and anti-polymerase I and transcript release factor,

in a biological sample originating from a patient, at various times, an increase in the amount of said at least one antibody over time being indicative of a worsening of SSc and/or of PAH.

Another aspect of the invention is an in vitro method for evaluating the efficacy of a treatment for SSc and/or PAH, which comprises determining the presence and/or the amount of at least one antibody selected from the group consisting of the following antibodies: anti-serum albumin precursor, anti-zyxin, anti-galectin-1, anti-ubiquitin carboxyl-terminal hydrolase isozyme L1, anti-caldesmon, anti-FAM10A4 protein, anti-Far-upstream element-binding protein 2, anti-cytoplasmic actin 2, anti-γ-enolase, anti-protein disulfide-isomerase A3 precursor, anti-desmin, anti-peripherin, anti-heterogeneous nuclear ribonucleoprotein H, anti-stress-induced phosphoprotein 1, anti-triosephosphate isomerase, anti-peroxiredoxin-6, anti-reticulocalbin-1, anti-peroxiredoxin-2, anti-thioredoxin-dependent peroxide reductase mitochondrial precursor, anti-Ran-specific GTPase-activating protein, anti-high mobility group protein B1, anti-tubulin beta-chain and anti-polymerase I and transcript release factor,

in a biological sample originating from a patient, at various times before, during or after the treatment, a decrease in the amount of said at least one antibody over time being indicative of an improvement in the SSc and/or in the PAH.

### DETAILED DESCRIPTION OF THE INVENTION

Systemic scleroderma (SSc) and pulmonary arterial hypertension (PAH) are both characterized by the presence of autoantibodies (AAbs) in the serum of patients, in particular AAbs directed against endothelial cells and fibroblasts. Before the studies presented below, no AAb directed against vascular smooth muscle cells (VSMCs) had been demonstrated. The characterization, by the inventors, of such antibodies directed against VSMCs is of major importance, in particular with regard to the key role of these cells in the physiopathology of SSc, with or without PAH, and of iPAH.

The inventors set out to identify the antibodies directed against VSMCs and to characterize the antigenic targets thereof. To do this, the inventors used VSMCs from internal mammary arteries as a source of antigens and tested sera from patients of identical phenotype (patients having SSc with or without PAH, patients having idiopathic PAH, or iPAH, and healthy individuals). The antibodies were investigated using a one- then two-dimensional immunoblotting technique followed by identification of the antigens by mass spectrometry (see the “examples” section below).

The inventors were thus able to demonstrate numerous IgG reactivities, some of which were very intense, with all the patient sera tested by one-dimensional immunoblotting, while the healthy individuals expressed virtually no IgG reactivity. The inventors characterized, by two-dimensional immunoblotting, several protein spots recognized by at least 80% of the IgGs of the pools of sera of a group of given patients, and not by the serum IgGs of a pool of healthy individuals, and other protein spots recognized by the vast majority of the serum IgGs of the pools of patients with a stronger intensity than that of the serum IgGs of the pool of healthy individuals.

### DEFINITIONS

The term “biological sample” refers to any biological sample originating from a patient. Examples of samples include biological fluids and tissue biopsies. The fibroblasts of scleroderma patients, cultured from skin biopsies, also constitute an example of a biological sample. Preferably, the sample may be blood, serum, saliva, urine or sperm. More preferably, the biological sample is a blood or serum sample.

The term “patient” refers to any individual capable of being tested. Preferably, it is a human being, but the term includes any other mammal, such as dogs, cats, rodents, cattle, horses, monkeys, etc. The patient can be tested irrespective of the sex or age thereof. The patient may be an individual at risk, may be asymptomatic or may show early or advanced signs of SSc and/or of PAH. For example, the patient may be an individual predisposed to developing SSc and/or PAH, in particular an individual carrying one or more mutations in the gene encoding BMPRII, endoglin or ALK1.

The term “diagnosis” means the identification of the pathological condition or the evaluation of the state of severity of the pathological condition.

The term “prognosis” means the evaluation of the risk of worsening, and of the consequences thereof.

The term “control value” refers to a basal value corresponding to the average of the values obtained with the biological sample from healthy individuals not suffering from SSc or PAH or a disease capable of leading to PAH. It may be a statistical reference value.

In order to evaluate the progression of the pathological condition, it may be useful to test a patient and to verify the effect of a treatment or the progression of the pathological condition by testing the patient again, for example with a gap of several months. In this case, the results of the second test are compared with the results of the first test, and also often with the “control” value.

An amount of antibodies “greater than the control value” generally means a statistically significant increase, for example of at least two standard deviations above the mean of the optical densities of the IgG reactivities of all the healthy individuals.

The term “capture antigen” is intended to mean an antigen, preferably attached to a solid phase, which is capable of retaining said at least one antibody present in a biological sample, by affinity binding. The capture antigen may be labeled.

The term “labeled” refers both to a direct labeling (by means of enzymes, radioisotopes, fluorochromes, luminescent compounds, etc.) and to an indirect labeling (for example by means of antibodies which are themselves directly labeled or using reagents of a labeled “affinity pair”, such as, but nonexclusively, the labeled avidin-biotin pair, etc.).

### Antibodies Identified:

As indicated in the “examples” section, the inventors identified several anti-VSMC antibodies in patients having SSc with or without PAH, or having iPAH.

The detection and/or the quantification of these antibodies can be carried out in order to detect SSc and/or PAH, in order to give the prognosis for or carry out the monitoring of these pathological conditions, or in order to evaluate the efficacy of a treatment for these pathological conditions.

The antigens recognized by the antibodies identified are listed below. The name and the accession numbers corresponding to these antigens in the SWISSPROT protein sequence database are given in tables 1 and 2 of the “examples” section.

The inventors characterized several reactivities against VSMCs in the sera from patients which are not found in the sera from healthy individuals. The antibodies identified are anti-78 kDa glucose-regulated protein precursor, anti-caldesmon, anti-FAM10A4 protein, anti-zyxin, anti-galectin-1, anti-protein disulfide-isomerase A3 precursor, anti-desmin, anti-peripherin, anti-heterogeneous nuclear ribonucleoprotein H, anti-tubulin beta-chain and anti-polymerase I and transcript release factor antibodies. In one particular embodiment, the method according to the invention comprises determining the presence of at least one antibody selected from the group consisting of the following antibodies: anti-78 kDa glucose-regulated protein precursor, anti-caldesmon, anti-FAM10A4 protein, anti-zyxin, anti-galectin-1, anti-protein disulfide-isomerase A3 precursor, anti-desmin, anti-peripherin, anti-heterogeneous nuclear ribonucleoprotein H, anti-tubulin beta-chain and anti-polymerase I and transcript release factor, in a biological sample originating from a patient, the presence of said at least one antibody being an indicator of systemic scleroderma and/or of pulmonary arterial hypertension, or of a risk of developing systemic scleroderma and/or pulmonary arterial hypertension.

The inventors also characterized several reactivities which have a significantly stronger intensity in the patients than in the healthy individuals. These reactivities correspond to anti-vimentin, anti-stress-induced phosphoprotein 1, anti-α-enolase, anti-triosephosphate isomerase, anti-serum albumin precursor, anti-ubiquitin carboxyl-terminal hydrolase isozyme L1, anti-cytoplasmic actin 2, anti-peroxiredoxin-6, anti-Far-upstream element-binding protein 2 (or anti-protein 2), anti-reticulocalbin-precursor, anti-γ-enolase, anti-thioredoxin-dependent peroxide reductase mitochondrial precursor, anti-Ran-specific GTPase-activating protein and anti-high mobility group protein B1 antibodies. In one particular embodiment, the antibodies corresponding to the reactivities which have a stronger intensity in the patients than in the healthy individuals are used in a method according to the invention, which comprises comparing the amount of at least one antibody selected from the group consisting of the following antibodies: anti-vimentin, anti-stress-induced phosphoprotein 1, anti-α-enolase, anti-triosephosphate isomerase, anti-serum albumin precursor, anti-ubiquitin carboxyl-terminal hydrolase isozyme L1, anti-cytoplasmic actin 2, anti-peroxiredoxin-6, anti-Far-upstream element-binding protein 2 (or anti-protein 2), anti-reticulocalbin-1 precursor, anti-γ-enolase, anti-thioredoxin-dependent peroxide reductase mitochondrial precursor, anti-Ran-specific GTPase-activating protein and anti-high mobility group protein B1, in a biological sample originating from a patient, with a control value, the presence of said at least one antibody in an amount greater than the control value being an indicator of systemic scleroderma and/or of pulmonary arterial hypertension, or of a risk of developing systemic scleroderma and/or pulmonary arterial hypertension.

The invention therefore relates to the use of at least one antibody directed against VSMCs, in a method, preferably an in vitro method, for detecting SSc and/or PAH.

The invention also relates to the use of at least one antibody directed against VSMCs, in a method, preferably an in vitro method, for giving the prognosis for or carrying out the monitoring of SSc and/or PAH.

The invention also relates to the use of at least one antibody directed against VSMCs, in a method, preferably an in vitro method, for evaluating the efficacy of a treatment for SSc or PAH.

Preferably, the antibodies directed against the VSMCs used in the methods of the invention are selected from the group consisting of the following antibodies: anti-serum albumin precursor, anti-zyxin, anti-galectin-1, anti-ubiquitin carboxyl-terminal hydrolase isozyme L1, anti-caldesmon, anti-FAM10A4 protein, anti-Far-upstream element-binding protein 2, anti-cytoplasmic actin 2, anti-γ-enolase, anti-protein disulfide-isomerase A3 precursor, anti-desmin, anti-peripherin, anti-heterogeneous nuclear ribonucleoprotein H, anti-stress-induced phosphoprotein 1, anti-triosephosphate isomerase, anti-peroxiredoxin-6, anti-reticulocalbin-1, anti-peroxiredoxin-2, anti-thioredoxin-dependent peroxide reductase mitochondrial precursor, anti-Ran-specific GTPase-activating protein, anti-high mobility group protein B1, anti-tubulin beta-chain and anti-polymerase I and transcript release factor.

In one particular embodiment, the antibodies directed against VSMCs used in the methods of the invention are selected from the group consisting of the following antibodies: anti-serum albumin precursor, anti-zyxin, anti-galectin-1, anti-ubiquitin carboxyl-terminal hydrolase isozyme L1, anti-caldesmon, anti-FAM10A4 protein, anti-Far-upstream element-binding protein 2, anti-cytoplasmic actin 2, anti-γ-enolase, anti-protein disulfide-isomerase A3 precursor, anti-desmin, anti-peripherin, anti-heterogeneous nuclear ribonucleoprotein H, anti-stress-induced phosphoprotein 1, anti-triosephosphate isomerase, anti-peroxiredoxin-6 and anti-reticulocalbin-1.

The antibodies identified by the inventors can be used in the methods according to the invention alone or in combination. The detection and/or the quantification can be carried out with respect to just one of the antibodies identified, or can concern a plurality of antibodies. It is thus possible to imagine carrying out the method on a solid support, for example a microplate, on which the antigens corresponding to the plurality of antibodies to be detected and/or quantified are arranged in a defined and ordered manner.

According to one embodiment of the invention, the methods described implement the detection of an anti-galectin-1 or anti-stress-induced phosphoprotein 1 antibody.

According to one embodiment of the invention, the methods described implement the detection of an anti-galectin-1 antibody for the diagnosis, prognosis or monitoring of SSc. This is because the inventors were able to show that this antibody is specific for SSc, in view of its presence in the sera of SSc patients with or without associated PAH, and its absence in the sera of patients suffering from iPAH.

According to another embodiment of the invention, the methods described implement the detection of an anti-78 kDa glucose-regulated protein precursor antibody for the diagnosis, prognosis or monitoring of SSc or of iPAH.

According to another embodiment of the invention, the methods described implement the detection of an anti-FAM10A4 protein antibody for the diagnosis, prognosis or monitoring of PAH, regardless of whether it is idiopathic or SSc-associated.

### Assaying of Antibodies:

The biological sample is preferably a serum sample, preferably diluted to 1/100th, or more, for example to 1/200th or 1/400th.

Advantageously, the amount of antibody can be determined by an immunoassay.

The biological sample can be optionally treated in a prior step, or brought directly into contact with at least one capture antigen.

The method according to the invention can be carried out according to various formats well known to those skilled in the art: in solid phase or in homogeneous phase; in one step or in two steps; in a competition method, by way of nonlimiting examples.

According to one preferred embodiment, the capture antigen is immobilized on a solid phase. By way of nonlimiting examples of a solid phase, use may be made of microplates, in particular polystyrene microplates, such as those sold by the company Nunc, Denmark. Use may also be made of solid particles or beads, paramagnetic beads, such as those provided by Dynal or Merck-Eurolab (France) (under the trademark Estapor™), or else polystyrene or polypropylene test tubes, etc.

An immunoassay format for detecting antibodies by competition is also possible. Other immunoassay modes can also be envisioned and are well known to those skilled in the art.

ELISA assays, radioimmunoassays, or any other detection technique can be used for revealing the presence of the antigen-antibody complexes formed.

According to one particular preferred embodiment, the capture antigen corresponds to a whole protein or to a fragment of said protein. For example, the method of the invention comprises bringing a biological sample into contact with a whole protein recognized by the antibody to be detected and/or quantified. By way of illustration, the invention comprises bringing a blood or serum sample into contact with whole galectin-1 or stress-induced phosphoprotein 1, for detecting and/or quantifying anti-galectin or anti-stress-induced phosphoprotein 1 antibodies in said sample.

In one particular example, the capture antigen may be coupled to a glutathione S transferase (GST), before being deposited on a microplate.

By way of illustration, the serum samples to be tested, for example diluted to 1/100th, are incubated on the microplate. After washing, labeled anti-human Fcγ antibodies (for example labeled with an alkaline phosphatase) are added, the complexes being revealed (for example by adding a phosphatase substrate, the cleavage of which can be detected by reading the absorbance).

### Patients Targeted:

The patients targeted are those to be likely to develop SSc and/or PAH.

This may involve a patient who suffers from PAH associated with a connective tissue disease, such as systemic scleroderma, Sharp's syndrome (which is a mixed connectivity) or systemic lupus erythematosis.

The patient may also be suffering from idiopathic or familial PAH.

More generally, any patient suffering from a pulmonary vascular disease can be advantageously subjected to the method for detecting PAH as defined in the invention.

Moreover, the PAH detected may also be portopulmonary hypertension (i.e. PAH associated with portal hypertension), or be associated with a congenital heart disease, or with a human immunodeficiency virus (HIV) infection, or else be post-embolic pulmonary hypertension, complicating the progression of chronic obstructive bronchitis or of cyanogenic heart disease.

Other patients targeted are those exposed to certain appetite-suppressing drugs, such as fenfluramine, the prescription of which can contribute to the occurrence of PAH.

Other individuals who may benefit from this type of test are those carrying a mutation in the gene encoding BMPRII, endoglin or ALK1, and who possibly do not present PAH detectable by echography, so as to screen for individuals who may subsequently develop PAH.

### Evaluation of the Efficacy of a Treatment:

Another aspect of the invention is an in vitro method for evaluating the efficacy of a treatment for SSc and/or PAH, which comprises determining the presence and/or the amount of at least one antibody as defined above in a biological sample originating from a patient, at various times before, during or after the treatment, a decrease in the amount of said at least one antibody over time being indicative of an improvement in the SSc or in the PAH.

The current conventional treatment for PAH combines symptomatic treatment and a vasodilator treatment. The symptomatic treatment combines anticoagulants, oxygen therapy and diuretics. The vasodilator treatment is based on the following molecules: calcium channel blockers, epoprostenol (prostacyclin) prescribed intravenously as a continuous infusion, selective or nonselective endothelin receptor inhibitors, in particular bosentan, sytaxentan and ambrysentan, phosphodiesterase type 5 inhibitors, in particular sildenafil and taladafil, all these medicaments being administered orally, and inhaled iloprost, a prostacyclin analog which is administered by inhalation. These treatments can be optionally combined. In the event of these therapies failing, a lung or heart-lung transplant can be proposed. During SSc, it is conventional to prescribe vasodilators, firstly calcium inhibitors in the treatment of Raynaud's phenomenon, proton pump inhibitors and a prokinetic, domperidone, in the treatment of gastroesophageal reflux. The other treatments depend on the ailments presented by the patient: colchicine or corticoids at low dose in the event of an inflammatory joint ailment, converting enzyme inhibitors in the event of renal crisis, cyclophosphamide in the event of evolving diffuse infiltrative lung disease, vasodilator treatment for pulmonary arterial hypertension.

The following figures and examples illustrate the invention without limiting the scope thereof.

### FIGURE LEGENDS

FIG. 1 corresponds to a one-dimensional immunoblot showing the reactivities of the serum IgGs of scleroderma patients with (n=3) or without (n=6) PAH, of patients having idiopathic PAH (n=6) and of healthy individuals (n=4) with respect to vascular smooth muscle cell proteins. PAH was documented by right catheterization in all the patients. SSc: systemic scleroderma; PAH: pulmonary arterial hypertension; iPAH: idiopathic pulmonary arterial hypertension; PAH-SSc: pulmonary arterial hypertension associated with scleroderma; C: internal control (PAH-SSc); PBS: phosphate buffered saline.

FIG. 2 corresponds to a two-dimensional reference gel of a total protein extract of vascular smooth muscle cells, stained with silver nitrate. First dimension (horizontal axis): pH 3-10, second dimension (vertical axis): 7-18% acrylamide gradient, allowing the counting of 880 protein spots.

FIG. 3 is a graph showing the number of protein spots recognized by the IgGs of 15 pools of 3 sera of phenotypically identical patients having systemic scleroderma and/or PAH and by a pool of 12 healthy individuals after adjustment on the reference gel. The y-axis scale indicates the number of IgG reactivities.

FIG. 4 shows the proportion of the reactivity spots recognized by the IgGs of the sera of the pools of 3 patients within each group (recognized or not recognized by the healthy individuals). 20%, 40%, 60%, 80%, 100%: number of protein spots recognized, respectively, by 1/5, 2/5, 3/5, 4/5, 5/5 of the pools of patients in a given group.

FIG. 5 represents the number of spots recognized by the IgGs of the sera of the pools of patients of each group and not recognized by the sera of the healthy individuals. 20%, 40%, 60%, 80%, 100%: number of protein spots recognized, respectively, by 1/5, 2/5, 3/5, 4/5, 5/5 of the pools of patients in a given group.

FIG. 6 shows the location of the candidate protein spots on the two-dimensional electrophoresis gel of a total protein extract of vascular smooth muscle cells, stained with silver nitrate. First dimension (horizontal axis): pH 3-10, second dimension (vertical axis): 7-18% acrylamide gradient.

FIG. 7 shows the intensity of the reactivities of the IgGs of the 15 pools of 3 sera of patients and of the pool of sera of healthy individuals, directed against α-enolase and stress-induced phosphoprotein 1, on PVDF membranes, of the various groups of patients. The area of PVDF membrane represented for each of the groups corresponds to a pHi of between 6.6 and 7.9 and MWs of between 51 and 70 kDa.

FIG. 8 is a graphic representation of the detection of anti-stress-induced phosphoprotein 1 (STIP1) antibodies by ELISA in the sera of patients suffering from SSc, iPAH and PAH associated with SSc, and in the sera of healthy control individuals (HC). The data reported correspond to the optical density of the proteins at 405 nm (OD₄₀₅), the background noise (OD₄₀₅ of the bicarbonate buffer) having been subtracted. Each point represents the reactivity of a serum sample. The single horizontal bars indicate the mean and the double horizontal bars indicate the standard deviation. The samples are considered to be positive when the optical density is greater than or equal to the mean+2 standard deviations of the control (2sd).

FIG. 9 shows the effect of the serum and of the purified IgGs of patients having SSc, SSc-PAH or iPAH, on the contraction of a collagen matrix by aortic VSMCs. The contraction of collagen matrices seeded with VSMCs was monitored for 4 days, in the presence of FCS, or of serum or purified IgGs of patients having SSc, SSc-PAH or iPAH. A, photographs of 4 matrices corresponding to the 4 conditions, incubated with the serum (A1) or the purified IgGs (A2), at D0 and D4. B, graphic representation of the kinetics of contraction of collagen matrices incubated with the serum (A1) or the purified IgGs (A2) of SSc, SSc-PAH or iPAH patients, or of healthy individuals, paired. For each condition, 10 sera or purified IgGs were used. *Sera: healthy/SSc p=0.012; purified IgGs: healthy/iPAH p=0.001.

FIG. 10 shows the effect of the serum and of the purified IgGs of patients having SSc, SSc-PAH or iPAH, on the contraction of a collagen matrix by VSMCs activated with TNF-α. The contraction of collagen matrices seeded with VSMCs was monitored for two (sera) or three (purified IgGs) days, in the presence of FCS, or of serum or purified IgGs of patients having SSc, SSc-PAH or iPAH. A: photographs of 4 matrices corresponding to the 4 conditions, incubated with the serum (A1) or the purified IgGs (A2), at D0 and D2 (serum) or D3 (purified IgGs). B: graphic representation of the kinetics of contraction of collagen matrices incubated with the serum (B1) or the purified IgGs (B2) of SSc, SSc-PAH or iPAH patients, or of healthy individuals, paired. For each condition, 10 sera or purified IgGs were used. *Healthy/iPAH p=0.001; healthy/SSc-PAH p=0.029.

### EXAMPLE 1

### Materials and Methods

Sera

The inventors used the sera of patients having iPAH or SSc with or without PAH. PAH was screened for by transthoracic echocardiography and confirmed by right catheterization. The scleroderma patients corresponded to the criteria of the American Rheumatology Association (ARA) and/or to the criteria of Leroy and Medsger. The sera were collected and stored at −80° C. before their use. All the patients had signed an informed consent in the context of the PAH-Ig study (Clinical Research and Investigation Contract 2005 No. CRC 05066, promoter Assistance Publique-HÔpitaux de Paris [Health and Social Security-Paris Hospitals]). Firstly, the sera of 15 patients having iPAH, 15 patients having PAH-SSc, 15 patients having SSc without PAH and 12 healthy individuals were tested in 1D immunoblotting experiments. Secondly, the same sera were tested in the form of pools of 3 sera of patients having a similar phenotype. A pool of 12 sera of healthy individuals, different than those used in 1D, was used as a control in the 2 D immunoblot experiments.

### Cells

Human VSMCs obtained from mammary arteries in patients having undergone an aortocoronary bypass graft were supplied to us by Dr Babett Weksler (Institut Cochin, Paris). These cells were immortalized after sequential lentiviral transduction of the catalytic subunit of the human holoenzyme telomerase reverse transcriptase and of the SV40 (Simian Virus 40) polyomavirus T antigen in a primary culture of adult VSMCs (Weksler et al., 2005). These cells were cultured in 175 cm² flasks in Smooth Muscle Cell Growth Medium 2 culture medium (PromoCell, Heidelberg, Germany) supplemented with 5% of decomplemented fetal calf serum (FCS), 0.5 ng/ml of Epithelial Growth Factor (EGF), 2 ng/ml of basic Fibroblast Growth Factor, 5 μg/ml of insulin, 1% of penicillin/streptomycin and 1% of ciprofloxacin. They were used for the 1D and 2D immunoblotting experiments.

Human aortic VSMCs (Cambrex) were used in the experiments evaluating the effect of the serum and of the purified IgGs of patients having SSc, SSc-PAH or iPAH on the contraction of a collagen matrix by nonactivated VSMCs or VSMCs activated with TNF-α.

Protein Extraction

### One-Dimensional Electrophoresis

The VSMCs that had reached confluence were detached with trypsin, washed with phosphate buffered saline (PBS) and then centrifuged at 1600 rpm at 20° C. The cell pellet was then recovered in a buffer containing 2% of sodium dodecyl sulfate (SDS), 62.5 mM Tris, pH 6.8, 5% 3-mercaptoethanol in the presence of protease inhibitors: 1 μg/ml of pepstatin, of aprotin and of leupeptin and 1 mM of phenylmethylsulfonyl fluoride (PMSF). The mixture was then sonicated 4 times for 30 sec in ice at 4° C. and at a power of 25 W, then heated for 10 min at 100° C. The protein extracts were then aliquoted and stored at −80° C. until use.

### Two-Dimensional Electrophoresis

The VSMCs that had reached confluence were washed twice in PBS without Mg²⁺ and Ca²⁺, and then detached and recovered in an isotonic solution in the absence of enzyme, containing chelating agents such as EDTA (Cell Dissociation Buffer enzyme free PBS-based, Invitrogen, Carlsbad, Calif., United States (US)). The cells were harvested and then centrifuged for 5 min at 1300 rpm at 20° C. After washing in an isotonic NaCl solution, a second centrifugation was carried out. After having repeated this operation a second time, the cell pellet was frozen at −80° C. in the presence of 1 mM of PMSF and of a cocktail of protease inhibitors (Complete Mini, Roche Diagnostic, Meylan, France).

In a second step, the proteins were extracted after three sonications, each for 30 s at 4° C. in a buffer composed of 5M urea, 2M thiourea, 2% 3-[(3-cholamidopropyl) dimethylammonio]-1-propane sulfonate (CHAPS), 40 mM Tris and 0.2% Bio-Lyte 3/10 ampholytes (ReadyPrep Sequential Extraction Reagent 3, Bio-Rad, Hercules, Calif., US). Two ultracentrifugations, each at 150 000 g for 25 min, were then carried out at 4° C. (Optima LE-80K, Beckman, Fullerton, Calif., US). In order to avoid artefactual disruption of the DNA released during the sonication, freezing at −80° C. was carried out in order to cause the DNA to precipitate. The extract was then thawed, and centrifuged and the supernatant recovered. Finally, the protein concentration was measured by the Lowry method (RC DC Protein Assay, Bio-Rad, Richmond, US). Dithiothreitol (DTT) was added to the extract at a final concentration of 64 mM before freezing at −80° C.

1D Immunoblotting

Protein Separation by One-Dimensional Electrophoresis The proteins of the sample were separated according to their molecular weight (MW) on denaturing polyacrylamide gels in the presence of SDS (SDS-PAGE) containing 10% of acrylamide (10% acrylamide, 0.27% bisacrylamide, 0.375 M Tris/HCI, pH 8.8, 0.1% SDS, 0.1% ammonium persulfate, 0.04% of tetramethylethylenediamine (TEMED) (Biorad, Hercules, Calif., US)). One hundred and twenty microliters of proteins were loaded at the top of each gel and the migration was carried out in a migration buffer (25 mM Tris/HCI, 192 mM glycine, 0.1% SDS) at 25 mA per gel at constant amperage with a mini-PROTEAN III device (Bio Rad) for approximately 50 min.
Electroblotting from One-Dimensional Gels

The proteins thus separated were transferred from the gel to a nitrocellulose membrane (Immunetics Inc., Boston, Mass., US) by means of a semi-dry electroblotting module (Semi Dry Electroblotter A ANCOS, Hoejby, Denmark) for 1 h at 50 mA per blotting module. The membranes were then blocked for 1 h 30 in PBS containing 0.2% Tween 20 (Sigma) and incubated overnight in the presence of sera belonging to one of the following three groups: SSc associated or not associated with PAH, iPAH. The sera of 12 healthy individuals were used as controls and PBS-0.2% Tween alone without Ab had been used as a negative control. Each patient serum was diluted to ½ in PBS-0.2% Tween and the sera of healthy individuals were diluted to 1/100.

After 5 short washes for 20 s and 5 long washes for 5 min in a solution of PBS-0.2% Tween, the membranes were incubated for 1 h 30 at 20° C. with an antihuman IgG secondary Ab specific for the human Fcγ fragment (anti-human Fcγ Ab) conjugated to alkaline phosphatase (Dako, Glostrup, Denmark). After 5 short washes and 1 long wash in PBS-0.2% Tween, the membranes were washed in a solution of tris buffered saline buffer (TBS: 24 mM Tris, 136.9 mM NaCl, 18.6 mM KCl, pH 8) and the reactivities were revealed using the substrate for alkaline phosphatase (bromochloroindolyl phosphate and nitroblue tetrazolium (Sigma)) in a buffer containing 100 mM Tris, 100 mM NaCl and 5 mM MgCl₂ (VWR International). The reaction was stopped by washing with double-distilled water, and the membranes were dried and then scanned using a high-resolution scanner (Perfection 1200S, Seiko Epson Corporation, Hirooka, Japan).

2D Immunoblotting

### Isoelectric Focusing (IEF)

IEF makes it possible to separate proteins according to their isoelectric pH (pHi). This step was carried out in an immobilized pH gradient (IPG), i.e. it was performed on an acrylamide gel poured on a rigid strip in which a pH gradient had been preformed, in this case a gradient of 3 to 10 (ReadyStrip 17 cm, pH 3-10, Bio-Rad). The strips were placed in a Bio-Rad horizontal tank of Protean IEF cell type at ambient temperature. Each strip was placed in a groove in the presence of a mixture containing rehydration buffer and 100 μg of VSMC protein extracts; the whole was covered with 2 ml of mineral oil in order to limit evaporation. The rehydration buffer consisted of 7M ultra-pure urea (VWR, Fontenay-Sous-Bois, France), 2M thiourea (Sigma), 4% CHAPS (Sigma), 0.002% triton X100 (Sigma), DTT (Sigma), bromophenol blue and Pharmalyte 3-10 ampholytes (Amersham Biosciences, Uppsala, Sweden). The IEF comprised passive hydration of the strips for 9 h, followed by active hydration of the strips for 12 h under a voltage of 50 V. Next, the IEF was carried out as follows: 1 h at 200 V (elimination of the excess salts), then a linear increase in the voltage for 1 h up to 1000 V, then for 6 h up to 10 000 V, then for 1 h up to 10 000 V.

### Acrylamide Gel Protein Separation (SDS-PAGE)

This second step made it possible to separate the proteins according to their MW. Twelve gels of 20×20×0.1 cm with an acrylamide gradient of 7% to 18.5% were poured simultaneously in a multigel chamber (Protean Plus Multi-Casting Chamber, Bio-Rad) ensuring optimum reproducibility and allowing separation of proteins having a MW of between 10 and 250 kDa. Before performing the second dimension, the strips obtained at the end of the previous step were brought into contact with two equilibration buffers in order to reduce and alkalinize the sulfhydryl groups of the cysteines. The first equilibration buffer was composed of 50 mM Tris, 6 mM urea, 40% glycerol, 52 mM SDS and 32.4 mM DTT. The second buffer was composed of 50 mM Tris, 6 mM urea, 40% glycerol, 52 mM SDS and 86.5 mM iodoacetamide. The strips were then kept in contact with the acrylamide gels in a 1% agarose solution (Ultrapure Low Melting Point Agarose, Gibco BRL Invitrogen) containing bromophenol blue in order to follow the migration front. MW markers had been placed on either side of the strip. The migration lasted approximately 30 h in a migration buffer (25 mM Tris, 192 mM glycine, 3.5 mM SDS, 1.25 mM sodium thiosulfate (Sigma)) maintained at 10° C. (Bio-Rad Protean Plus Dodeca Cell, Amersham Biosciences MultiTemp III Thermostatic Circulator) at constant amperage; 40 V for 1 h then 80 V for 1 h and, finally, 15 mA/gel until the migration front has exited the gels.

At the end of the migration in the second dimension, 11 gels were blotted on to polyvinylidene fluoride (PVDF) membranes (Immobilon-P Transfer Membranes, pores of 0.45 μm, Millipore, Bedford, Mass., US), while the last gel was stained with silver nitrate.

### Electroblotting

The semi-dry blotting was carried out at 4° C. for 1 h 30 at constant amperage (320 mA). At the end of the blotting, the membranes were immersed for 5 min in a solution of PBS composed of 148 mM NaCl, 3.5 mM NaH₂PO₄.2H₂O, 17.6 mM Na₂HPO₄.12H₂O, and then dried.

### Staining of Non-Blotted Gels

The non-blotted gel (also called reference gel) was stained with silver nitrate (Rabilloud et al., 1990) in 5 steps; fixing (30% absolute ethanol, 5% acetic acid), washing (11.8 mM silver nitrate (Sigma), 3.45 mM formaldehyde (Sigma)), staining (0.02% silver nitrate) and, finally, visualizing (37% formaldehyde, sodium carbonate, thiosulfate). The gel was then stored in a preserving solution (2% dimethyl sulfoxide (Sigma), 10% acetic acid) before being scanned using a densitometer (GS-800, Bio-Rad).

Incubation of the Pvdf Membranes with the Sera and Visualizing of Reactivities

The PVDF membranes initially blocked with PBS-0.2% Tween were incubated overnight in the presence of sera of patients belonging to one of the three groups described above: iPAH, PAH-SSc or SSc without PAH. For each membrane, a pool of three sera belonging to the same group, diluted to 1/100th in a solution of PBS-0.2% Tween, was used. For each experiment, one membrane was incubated with a pool of 12 sera of healthy individuals, diluted to 1/100th in the same buffer. The visualizing of the reactivities was carried out as previously in the case of the 1D immunoblotting (anti-human Fcγ Ab conjugated to alkaline phosphatase, revealing the reactivities using the substrate for alkaline phosphatase) and then the membranes were dried and photographed using a densitometer (GS-800, Bio-Rad). The membranes were then stained with colloidal gold (Protogold®, BioCell, Cardiff, GB) in order to visualize all the blotted proteins at the surface of the membranes. A further densitometric acquisition was then carried out.

### Computer Analysis

The computer analysis of the gels and membranes was carried out using software specially designed for analyzing two-dimensional gels (Image Master 2D® Platinum 6.0, Buckinghamshire, England). The first step consisted of automatic detection of the protein spots according to the parameters that had been chosen (number of smoothings carried out in order to eliminate the background noise, Laplacian threshold and minimum surface area of the spots to be detected). The spots detected were controlled visually by means of three-dimensional reconstruction methods, so as to eliminate the false positives and to see the reactivity spots not detected by the software. Each protein spot recognized by the IgGs of an individual was then paired with the corresponding protein by means of the densitometric photograph of the same membrane taken after staining with colloidal gold. This step was carried out for each of the 16 membranes. Finally, the proteins blotted on to the membranes were paired with the proteins of the gel selected as reference gel. This made it possible to collect all the information on the reference gel and to be able to subsequently compare the protein spots recognized by the healthy individuals and the patients within the various groups studied.

### Mass Spectrometry

The protein spots recognized as antigenic targets were extracted by taking plugs from a new acrylamide gel loaded with 400 μg of protein extracts and stained with Coomassie blue. Each spot removed as a plug was placed in the well of a 96-well plate and digested in the presence of trypsin (Promega, France) overnight. The samples digested were then transferred on to another 96-well plate subsequently stored at 4° C. before analysis by Matrix-Assisted Laser Desorption Ionization-Time-of-Flight (MALDI-TOF) mass spectrometry (PerSeptive Biosystems, Framingham, Mass., US).

Assaying of anti-STIP1 antibodies by ELISA Stress-induced phosphoprotein 1 (STIP1) was obtained from the company Tebu-bio (Tebu-bio, Maryland, USA), diluted in a bicarbonate buffer and deposited on 96-well plates (Maxisorb, NalgeNunc Int. Rochester, N.Y., USA) at a final concentration of 3 μg/ml at 4° C. The reactivity of the serum IgGs obtained from 75 scleroderma patients without PAH, 74 suffering from iPAH, 37 scleroderma patients with PAH (SSc-PAH) and 70 healthy individuals (HC) were tested by ELISA against STIP-1. The wells were washed five times with phosphate buffer (PBS) and blocked using a PBS-1% bovine serum albumin solution for one hour at 37° C. The sera were diluted to 1/100th in PBS, introduced in duplicate and incubated for one hour at ambient temperature. Mouse anti-STIP-1 polyclonal antibodies (Tebu-bio, Maryland, USA) were diluted to 1/500th and used as a positive control. The plates were washed as mentioned above, and rabbit anti-human Fcγ antibodies conjugated to alkaline phosphatase (Tebu-bio, Maryland, USA; diluted to 1/1000th), with donkey anti-rabbit IgG antibodies (Jackson ImmunoResearch, West Baltimore Pike, USA; diluted to 1/10 000th), were incubated for one hour at ambient temperature. The reactivities were visualized by adding p-nitrophenylphosphate (Sigma-Aldrich, St. Louis, USA) and the absorbance (DO) at 405 nm was measured. The optical density background noise (wells covered with bicarbonate buffer only) was subtracted from the OD value obtained with the proteins. The samples were considered to be positive when the optical density was greater than or equal to the mean+2 standard deviations of the control (2sd).

### Study of the Effect of the Sera or of the Purified IgGs of Patients Versus Healthy Individuals on the Contraction of VSMCs or Fibroblasts

### Sera and Purified IgGs

The inventors used the sera of 10 scleroderma patients without PAH (subsequently referred to as SSc), 10 scleroderma patients with PAH (SSc-PAH) and 10 patients suffering from iPAH. Ten healthy individuals paired for sex and age were also tested. The scleroderma patients correspond to the criteria of the American Rheumatology Association (ARA) and/or to the criteria of Leroy and Medsger. The sera were stored at −80° C. before their use. All the patients and the healthy individuals signed an informed consent in the context of the PAH-Ig study (Clinical Research and Investigation Contract 2005 No. CRC 05066, promoter Assistance Publique-Hôpitaux de Paris [Health and Social Services-Paris Hospitals]; investigator-coordinator Luc Mouthon; management center URC Cochin).

The IgGs were purified from the serum of the patients and of the healthy individuals on a protein G sepharose column. The purified IgGs were quantified by spectrophotometry at 260 and 280 nm. The purity of the purified IgG preparations was attested to by SDS-PAGE.

### Cell Culture

Human VSMCs obtained from mammary arteries in patients having undergone an aortocoronary bypass graft, immortalized after sequential lentiviral transduction of the catalytic subunit of the human holoenzyme Telomerase Reverse Transcriptase (hTERT) and of the SV40 (Simian Virus 40) polyomavirus T antigen in a primary culture of adult VSMCs (Weksler et al. 2005), were provided by Dr Babett Weksler (Institut Cochin, Paris). These cells were cultured in DMEM culture medium (Gibco BRL Invitrogen™ Cergy Pontoise, France) supplemented with 10% of filtered and decomplemented fetal calf serum (FCS), with 1% of penicillin/streptomycin and 1% of ciprofloxacin.

Human aortic VSMCs (PromoCell, Heidelberg, Germany) were cultured in Smooth Muscle Cell Growth Medium 2 culture medium (PromoCell, Heidelberg, Germany) supplemented with 5% of decomplemented FCS, 0.5 ng/ml of Epithelial Growth Factor (EGF), 2 ng/ml of basic Fibroblast Growth Factor (bFGF), 5 μg/ml of insulin, 1% of penicillin/streptomycin and 1% of ciprofloxacin.

### Contraction of a Collagen Matrix

VSMCs were harvested with 0.25% trypsin, 1 mM EDTA (Gibco BRL Invitrogen™ Cergy Pontoise, France), neutralized with 5% FCS. The collagen matrices were prepared in 35 mm dishes with 1 ml of FCS-free medium containing 500 000 VSMCs, 1.65 ml of medium containing 1% of serum (FCS, patient serum or healthy serum) or 128 μg/ml of purified IgGs, and 1 ml of 3.35 mg/ml collagen (BD Biosciences, Franklin Lakes, US). For the contraction test with activation of the VSMCs, 40 ng/ml of TNF-α (R&D systems, Abingdon, England) were added. After incubation for 1 h at 37° C. allowing polymerization, the matrices were detached by tapping gently on the edges of the dish, in order to initiate the contraction. In order to determine the degree of contraction of the gel, photographs were taken on D2 and on D4, in order to measure the surface area of the matrix. The results obtained with the sera of healthy individuals and of patients were compared. 20 sera were tested in each experiment; the various contraction tests were calibrated using FCS and a reference serum used in duplicate. This control made it possible to verify the good reproducibility of the test. The measurements were carried out by means of the Image J software (National Institute of Health NIH, US).

### Results

### 1D Immunoblotting

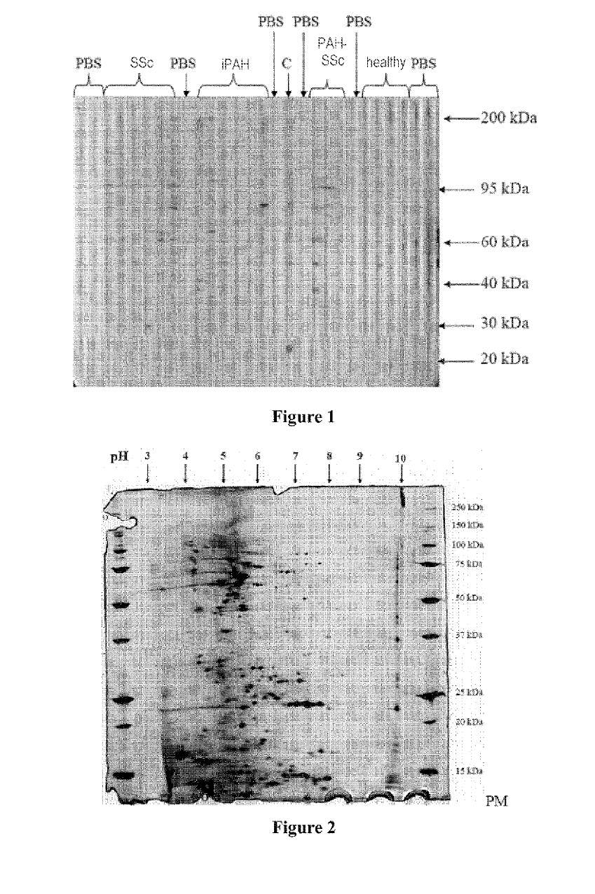

In a first step, the inventors separately tested the IgG reactivities of the sera of 15 patients in each group and of 15 healthy individuals by 1D immunoblotting at a dilution of 1/100. They demonstrated numerous reactivities with the sera from patients (SSc, PAH-SSc, iPAH), some of which were very intense in comparison with the sera from healthy individuals, which showed virtually no IgG immunoreactivity band. The number of reactivity bands was higher in the case of the scleroderma patients with or without PAH than in the case of the patients having iPAH. Furthermore, certain reactivity bands appeared to be specific for a given group of patients, in particular a band at approximately 90 kDa in certain scleroderma patients with or without PAH (FIG. 1). In order to identify the antigenic targets of the anti-VSMC IgGs of the patients, the inventors subsequently carried out 2D immunoblotting.

Mapping of VSMC Proteins after Two-Dimensional Separation

After having migrated 100 μg of VSMC protein extracts prepared as described in the Materials and Methods section, the inventors were able to separate and then stain with silver nitrate 880 protein spots and to obtain the gel represented in FIG. 2. The inventors were able to estimate the MW and the pHi of each of these protein spots after computer analysis according to how they were placed in the gel and by means of MW markers; they predominantly had an MW of between 10 and 125 kDa and a pHi of between 3 and 8.

2D Immunoblotting of the Serum IgGs of Healthy Individuals and of Patients with Respect to Vascular Smooth Muscle Cell Proteins

635 reactivities were identified after pairing of the reactivities present on each of the sixteen PVDF membranes with the reference gel, taking into consideration the reactivities of the serum IgGs of the pools of three patients and those of the pool of healthy individuals added.

Healthy Individuals

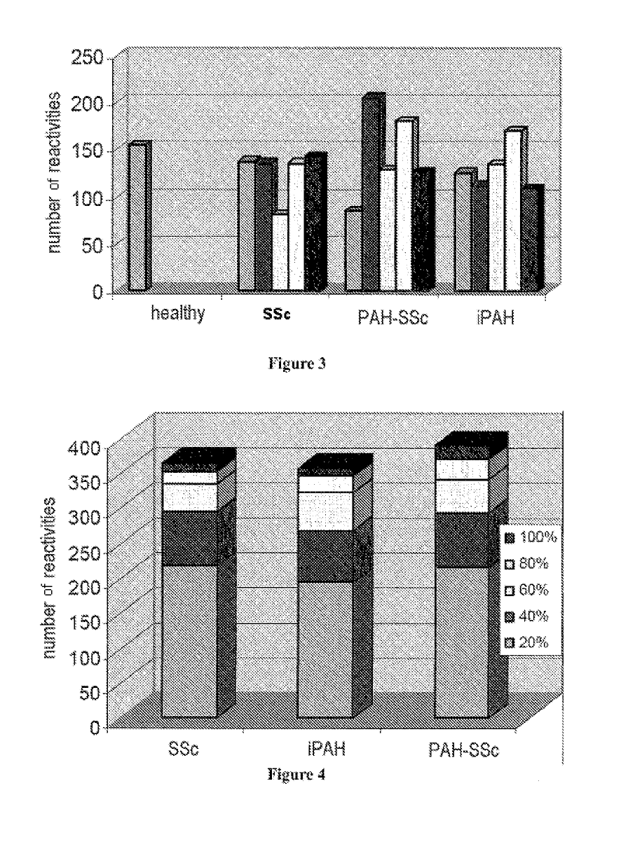

The sera of 12 healthy individuals were mixed and their reactivities were tested. The IgGs of these individuals recognized 150 VSMC protein spots (FIG. 3). Twenty-one protein spots were specific for healthy individuals (not recognized by the patients).

Scleroderma Patients

The 5 pools of 3 sera of patients suffering from SSc without PAH recognized on average 127±26 protein spots (FIG. 3) and a total of 367 different spots. 71% of these spots were not recognized by the healthy individuals. Among these 367 spots, 13 were common to the 5 pools of scleroderma patients (including just one not recognized by the healthy individuals), 18 were common to 4 pools out of 5 (including 7 not recognized by the healthy individuals) and 39 were common to 3 pools out of 5 (including 19 not recognized by the healthy individuals) (FIG. 4).

The protein spots common to the 5 pools of SSc patients without PAH were also all recognized by certain pools of patients suffering from iPAH and from PAH-SSc. Out of the 18 protein spots recognized by 4/5 of the pools of SSc patients, 9 were also recognized by at least 3/5 of the pools of patients of each of the other two groups of afflicted individuals (including 3 not recognized by the healthy individuals), 5 were recognized by at least 3/5 of the pools of PAH-SSc patients (including one not recognized by the healthy individuals) and 3 were recognized by at least 3/5 of the pools of iPAH patients (including 2 not recognized by the healthy individuals). One spot (5325) was recognized by just one pool of PAH-SSc patients, by no pool of iPAH patients and by no pool of healthy individuals.

The IgGs of the 5 pools of 3 sera of patients suffering from PAH-SSc recognized on average 145±48 protein spots (FIG. 4). In total, 264 different protein spots were recognized by the serum IgGs of these patients, including 77% not recognized by the IgGs of the healthy individuals. Among these 264 protein spots, 19 were common to the 5 pools of PAH-SSc patients (including 2 not recognized by the healthy individuals), 29 were common to 4/5 of the pools (including 9 not recognized by the healthy individuals) and 47 were common to 3/5 of the pools (including 30 not recognized by the healthy individuals) (FIG. 4).

The protein spots common to the 5 pools of PAH-SSc patients were also predominantly recognized by the patients suffering from iPAH and from PAH-SSc. More specifically, 16 spots were recognized by at least 3/5 of the pools of patients of the other two groups (including just 1 not recognized by the healthy individuals), 3 were recognized by at least 3/5 of the pools of iPAH patients (including just 1 not recognized by the healthy individuals) and 1 spot was recognized by at least 3/5 of the pools of SSc patients.

Contrary to the patients suffering from SSc, the majority of the spots recognized by 4/5 of the pools of PAH-SSc patients were recognized by less than 40% of the afflicted individuals of the other two groups. More specifically, 12 spots were recognized by less than 40% of the afflicted individuals of the other two groups, including 5 spots not recognized by the SSc patients (4658, 5206, 4831, 4707, 4659) and 3 spots not recognized by the iPAH patients (4656, 5190, 4707). 9 spots were recognized by at least 3/5 of the pools of SSc and iPAH patients (including 2 not recognized by the healthy individuals), 5 were recognized by at least 3/5 of the iPAH patients (including 1 not recognized by the healthy individuals) and 3 were recognized by at least 3/5 of the SSc patients (these 3 spots were also all recognized by the healthy individuals).

Patients Suffering from Idiopathic PAH

The IgGs of the 5 pools of 3 sera of patients suffering from iPAH recognized on average 130±25 protein spots (FIG. 3). In total, 356 different protein spots were recognized by the IgGs of these patients, and 70% were not recognized by the IgGs of the healthy individuals. Among these 356 protein spots, 12 were common to the 5 pools of patients (but all were recognized by the healthy individuals), 24 were common to 4/5 of the pools (including 7 not recognized by the healthy individuals) and 54 were common to 3/5 of the pools (including 31 not recognized by the healthy individuals) (FIG. 4).

The protein spots common to the 5 pools of iPAH patients were also predominantly recognized by pools of patients suffering from iPAH or PAH-SSc. More specifically, 10 were also recognized by at least 3/5 of the pools of SSc patients and of PAH-SSc patients, one was also recognized by at least 3/5 of the pools of SSc patients and one other by at least 3/5 of the pools of PAH-SSc patients.

The protein spots recognized by 4/5 of the pools of sera of iPAH patients were predominantly shared with the other two groups of afflicted individuals. More specifically, 15 spots were also recognized by at least 3/5 of the pools of SSc patients and 3/5 of the pools of PAH-SSc patients (including 4 not recognized by the healthy individuals), 3 spots were also recognized by at least 3/5 of the pools of SSc patients (including one not recognized by the healthy individuals) and one was also recognized by at least 3/5 of the pools of PAH-SSc patients (and by the healthy individuals). 5 spots were recognized by less than 40% of the pools of SSc or PAH-SSc patients (including 4 not recognized by the healthy individuals). Among these 4 spots, one was not recognized by the SSc patients (4735).

Comparison of the Reactivities of the IgGs of Scleroderma Patients with or without PAH, of Patients Having Idiopathic PAH and of Healthy Individuals and Identification of the Antigens Specific for a Group of Afflicted Individuals

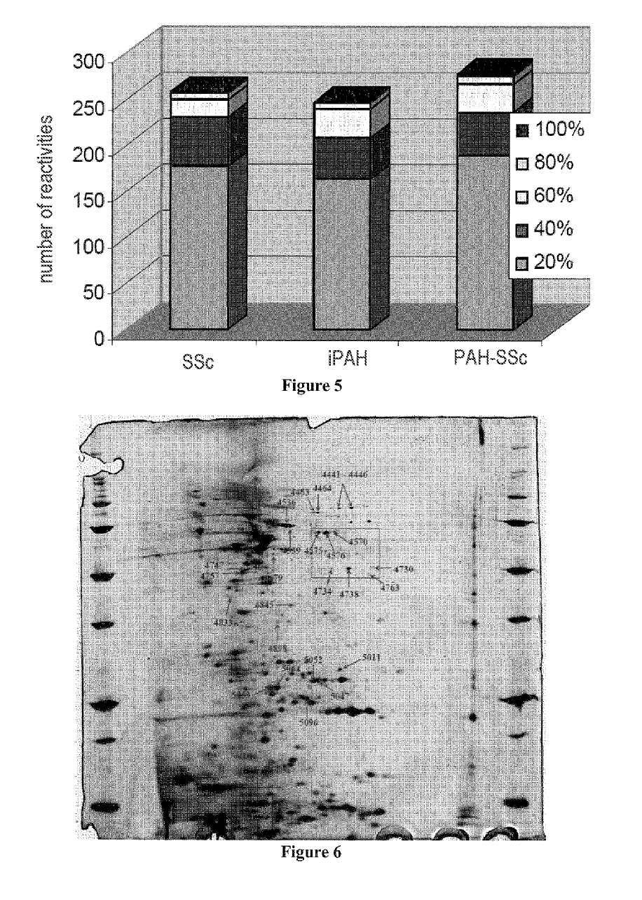

The inventors compared the IgG reactivity profiles of the pool of healthy individual sera and of the pools of patient sera with respect to VSMC proteins. Irrespective of the group of patients, most of the protein spots not recognized by the IgGs of healthy individuals were recognized by a single patient pool out of 5 (FIG. 5). By selecting the protein spots recognized by at least 3/5 of the pools of sera of patients of a given group and not by the pool of healthy individuals, the inventors identified 21 protein spots of interest (table 1). Even though the result of all the protein spots digested has not yet been obtained, it has been possible to identify 13 interesting protein spots.

The location of these protein spots on the reference gel is indicated in FIG. 6. Two spots (5190, 5325) appear to be SSc-specific since they are recognized, respectively, by 3/5 and 4/5 of the pools of SSc patients and 4/5 and 1/5 of the pools of PAH-SSc patients, but by no pool of iPAH patients. One of them (5325) was identified as being galectin.

TABLE 1
Identification of the protein spots recognized by the IgGs of at least 4/5 of the pools of
patients of a given group and not by the IgGs of the pool of healthy individuals. The identification of
the same candidate antigen for different spots corresponds to the detection of isoforms of the protein

| | | | | Number of pools of | | | |
|---|---|---|---|---|---|---|---|
| | | | | patients recognizing the | | | |
| | | | | antigen | | | Swissprot name and |
| | | | | | PAH- | | accession number of |
| | | | Candidate | SSc | SSc | iPAH | the candidate |
| Spot | pHi | MW(kDa) | antigen | (n = 5) | (n = 5) | (n = 5) | antigens |
| 4484 | 6.7 | 82 | 78 kDa glucose- | 4 | 0 | 3 | GRP78_HUMAN |
| | | | regulated protein | | | | P11021 |
| | | | precursor | | | | (SEQ ID NO: 18) |
| 4488 | 6.8 | 81 | Caldesmon | 2 | 2 | 4 | CALD1_HUMAN |
| | | | | | | | Q05682 |
| | | | | | | | (SEQ ID NO: 5) |
| 4735 | 5.5 | 51 | FAM10A4 protein | 0 | 2 | 4 | F10A4_HUMAN |
| | | | | | | | Q8IZP2 |
| | | | | | | | (SEQ ID NO: 6) |
| 4787 | 5.7 | 46 | Cytoplasmic actin | 3 | 3 | 4 | ACTG_HUMAN |
| | | | 2 | | | | P63261 |
| | | | | | | | (SEQ ID NO: 8) |
| 4660 | 6.2 | 60 | Protein disulfide- | 1 | 4 | 1 | PDIA3_HUMAN |
| | | | isomerase A3 | | | | P30101 |
| | | | precursor, | | | | (SEQ ID NO: 10) |
| | | | Desmin, | | | | DESM_HUMAN |
| | | | Peripherin | | | | P17661 |
| | | | | | | | (SEQ ID NO: 11) |
| | | | | | | | PERI_HUMAN |
| | | | | | | | P41219 |
| | | | | | | | (SEQ ID NO: 12) |
| 4691 | 6.4 | 56 | Heterogeneous | 1 | 4 | 1 | HNRH1_HUMAN |
| | | | nuclear | | | | P31943 |
| | | | ribonucleoprotein | | | | (SEQ ID NO: 13) |
| | | | H | | | | |

Identification of the Target Antigens of the Anti-VSMC IgGs Recognized with a Significantly Stronger Intensity in the Afflicted Individuals than in the Healthy Individuals

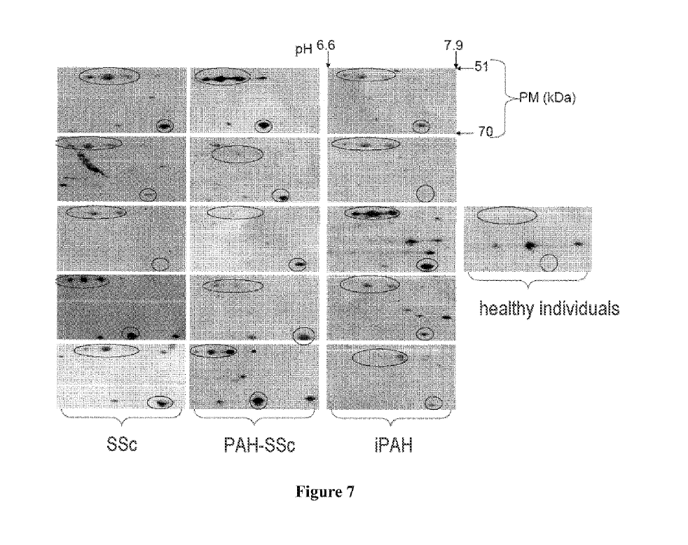

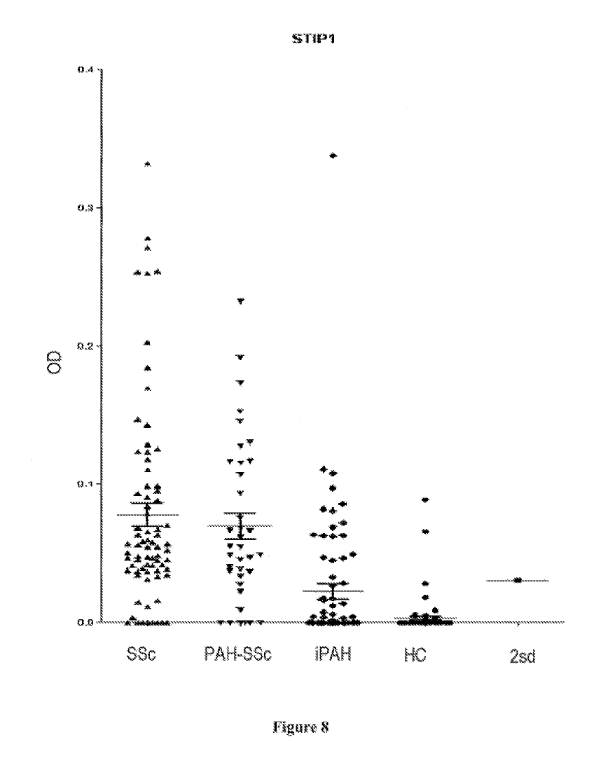

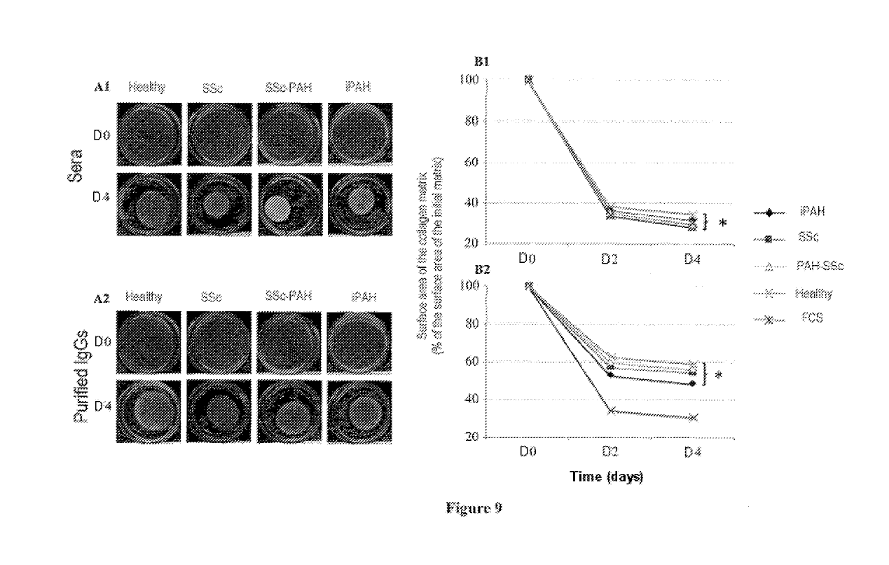

In a second step, the inventors identified the VSMC protein spots recognized by the IgGs of pools of 3 patient sera and by the IgGs of the pool of healthy individuals, with the condition that these protein spots are recognized with a strong intensity by the IgGs of a large number of pools of 3 patient sera and with a stronger intensity than the healthy individuals. Twenty-seven protein spots corresponded to these criteria (table 2). The reactivities of the serum IgGs of the various groups of afflicted individuals with respect to the protein spots 4576, 4570 and 4576 identified as isoforms of stress-induced phosphoprotein and with respect to the spot 4738 identified as α-enolase are represented in FIG. 7. The region selected is represented in FIG. 6.

TABLE 2
Identification of the protein spots recognized with a significantly stronger intensity in the
patients than in the healthy individuals

| | | | | Number of pools of | | | |
|---|---|---|---|---|---|---|---|
| | | | | patients recognizing the | | | |
| | | | | antigen | | | |
| | | | | | | PAH- | Swissprot name and |
| | | | Candidate | SSc | iPAH | SSc | accession number of the |
| Spot | pHi | MW(kDa) | antigen | (n = 5) | (n = 5) | (n = 5) | candidate antigens |
| 4757 | 5.2 | 49 | Vimentin | 5 | 4 | 5 | VIME_HUMAN |
| | | | | | | | P08670 |
| | | | | | | | (SEQ ID NO: 19) |
| 4576 | 6.9 | 70 | Stress-induced | 5 | 4 | 5 | STIP1_HUMAN |
| | | | phosphoprotein 1 | | | | P31948 |
| | | | | | | | (SEQ ID NO: 14) |
| 4570 | 7.1 | 70 | Stress-induced | 5 | 5 | 5 | STIP1_HUMAN |
| | | | phosphoprotein 1 | | | | P31948 |
| | | | | | | | (SEQ ID NO: 14) |
| 4575 | 6.8 | 70 | Stress-induced | 5 | 4 | 5 | STIP1_HUMAN |
| | | | phosphoprotein 1 | | | | P31948 |
| | | | | | | | (SEQ ID NO: 14) |
| 4738 | 7.4 | 51 | α-enolase | 5 | 5 | 5 | ENOA_HUMAN |
| | | | | | | | P06733 |
| | | | | | | | (SEQ ID NO: 20) |
| 5052 | 6.7 | 27 | Triosephosphate | 3 | 2 | 2 | TPIS_HUMAN |
| | | | isomerase | | | | P60174 |
| | | | | | | | (SEQ ID NO: 15) |
| 4536 | 6.1 | 74 | Serum albumin | 4 | 4 | 4 | ALBU_HUMAN |
| | | | precursor | | | | P02768 |
| | | | | | | | (SEQ ID NO: 1) |
| 5063 | 5.8 | 26 | Ubiquitin | 4 | 1 | 3 | UCHL1_HUMAN |
| | | | carboxyl-terminal | | | | P09936 |
| | | | hydrolase | | | | (SEQ ID NO: 4) |
| | | | isozyme L1 | | | | |
| 5064 | 5.9 | 26 | Ubiquitin | 4 | 1 | 4 | UCHL1_HUMAN |
| | | | carboxyl-terminal | | | | P09936 |
| | | | hydrolase | | | | (SEQ ID NO: 4) |
| | | | isozyme L1 | | | | |
| 4463 | 6.7 | 85 | Zyxin | 4 | 3 | 4 | ZYX_HUMAN |
| | | | | | | | Q15942 |
| | | | | | | | (SEQ ID NO: 2) |
| 4539 | 6.2 | 73 | Serum albumin | 4 | 4 | 4 | ALBU_HUMAN |
| | | | precursor | | | | P02768 |
| | | | | | | | (SEQ ID NO: 1) |
| 5325 | 5.4 | 15 | Galectin-1 | 4 | 0 | 1 | LEG1_HUMAN |
| | | | | | | | P09382 |
| | | | | | | | (SEQ ID NO: 3) |
| 4734 | 7.0 | 51 | α-enolase | 4 | 1 | 5 | ENOA_HUMAN |
| | | | | | | | P06733 |
| | | | | | | | (SEQ ID NO: 20) |
| 5047 | 6.8 | 27 | Peroxiredoxin-6 | 2 | 5 | 4 | PRDX6_HUMAN |
| | | | | | | | P30041 |
| | | | | | | | (SEQ ID NO: 16) |
| 4441 | 7.4 | 89 | Protein 2 (Far | 3 | 4 | 3 | FUBP2_HUMAN |
| | | | upstream | | | | Q92945 |
| | | | element-binding | | | | (SEQ ID NO: 7) |
| | | | protein 2) | | | | |
| 4446 | 7.2 | 89 | Protein 2 (Far | 3 | 4 | 2 | FUBP2_HUMAN |
| | | | upstream | | | | Q92945 |
| | | | element-binding | | | | (SEQ ID NO: 7) |
| | | | protein 2) | | | | |
| 4833 | 4.9 | 43 | Reticulocalbin-1 | 2 | 3 | 5 | RCN1_HUMAN |
| | | | precursor | | | | Q15293 |
| | | | | | | | (SEQ ID NO: 17) |
| 4747 | 5.2 | 50 | γ-enolase, | 1 | 3 | 5 | ENOG_HUMAN |
| | | | Vimentin | | | | P09104 |
| | | | | | | | (SEQ ID NO: 9) |
| | | | | | | | VIME_HUMAN |
| | | | | | | | P08670 |
| | | | | | | | (SEQ ID NO: 19) |

### ELISA Assay of Anti-STIP1 Antibodies

The inventors demonstrated that 56/75 (74.6%) scleroderma patients, 24/74 (32.4%) patients having iPAH, 27/37 (73%) patients having PAH-SSc and 2/70 (2.8%) healthy individuals had anti-STIP1 antibodies. Thus, close to three quarters of the scleroderma patients, irrespective of whether or not they had PAH, and close to a third of the patients suffering from iPAH, had anti-STIP1 Abs, whereas these antibodies were, as a general rule, absent in the healthy individuals.

### Effect of the Sera or of the Purified IgGs of Patients Versus Healthy Individuals on VSMC or Fibroblast Contraction

The inventors determined whether the sera and/or the serum IgGs of patients having SSc and/or PAH had an effect on VSMC and fibroblast contraction, a phenomenon involved in vascular remodeling and cell mobility. For this, the cells were seeded in a collagen matrix, and incubated in the presence of 1% of FCS, of sera or of purified IgGs of patients having SSc and/or PAH, versus those of healthy individuals. The quantifiable retraction of the collagen matrix reflects the contractile activity of the cells.

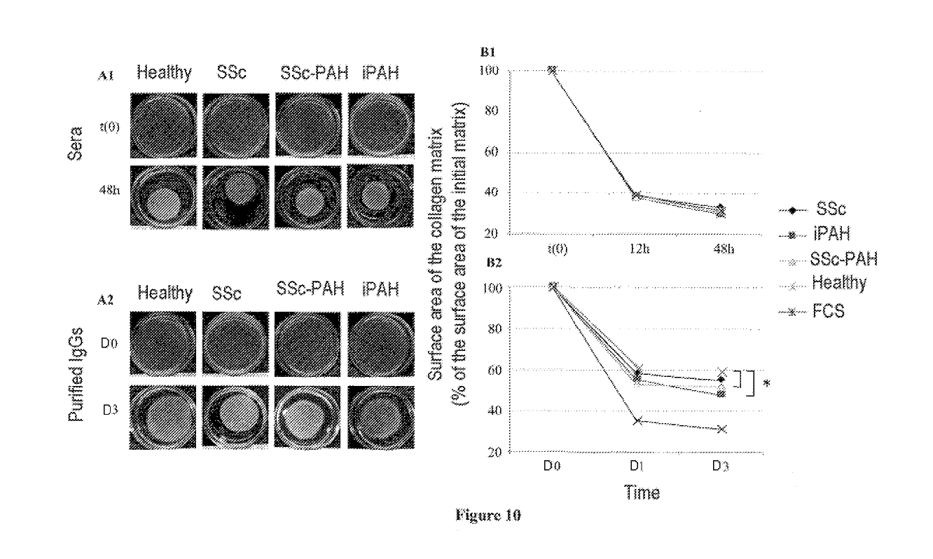

The experiment was carried out using healthy, nonactivated cells. The kinetics of contraction of the collagen matrices were monitored for 4 days for the VSMCs and 7 days for the fibroblasts. The results obtained with the VSMCs are given in FIG. 9.

### VSMCs

For these cells, the sera of 15 patients of each pathological condition (SSc, iPAH, SSc-PAH) and of 15 healthy individuals and also the purified IgGs of 10 of these 15 patients and of 10 of these 15 healthy individuals were tested. FCS, used in duplicate, made it possible to weight the various tests with respect to one another. The kinetics of contraction of the collagen matrices was monitored for 4 days, preliminary experiments having demonstrated that the modifications observed beyond this time were minimal (FIG. 9B); the surface areas of the collagen matrices were measured on D2 and D4 by means of the Image J software and calculated as percentage of the surface area of the initial matrix.

On D4, the mean of the surface areas of the 15 matrices (as % of the initial surface area) incubated with the serum was 27.8%±6.0 for the SSc patients, 31.1%±8.3 for the iPAH patients, 29.4%±4.7 for the SSc-PAH patients and 34.3%±7.1 for the healthy individuals. The 15 surface areas of the collagen matrices incubated with the serum of the SSc patients differed significantly compared with the surface areas of the 15 matrices incubated with the serum of the healthy individuals (p=0.012). On the other hand, the differences between the surface areas obtained in the presence of the other two groups of sera from patients (iPAH, SSc-PAH) and the group of sera from the healthy individuals were not significant.

On D4, the mean of the surface areas of the 10 matrices (as % of the initial surface area) incubated with the purified IgGs was 53.9%±8.2 for the SSc patients, 48.0%±3.2 for the iPAH patients, 55.8%±8.9 for the SSc-PAH patients and 34.3%±8.6 for the healthy individuals. A significant difference is noted between the matrices incubated with the purified IgGs of iPAH patients and those incubated with the IgGs of healthy individuals (p=0.001).

If the two experiments are compared with one another (FIG. 9B1 compared with 9B2), it is noted that the matrices incubated with the purified IgGs retracted less than those incubated with the sera.

### EXAMPLE 2

The inventors subjected the samples of the patients suffering from SSc, from PAH-SSc or from iPAH, and also the samples of healthy individuals, to another analysis of their reactivities in order to refine the results obtained and to identify other anti-VSMC antibodies.

This study made it possible to demonstrate reactivities against peroxiredoxin-2 (spot 5122; Swissprot: PRDX2_HUMAN, No. P32119; SEQ ID NO:21), thioredoxin-dependent peroxide reductase mitochondrial precursor (spot 5096; Swissprot: PRDX3_HUMAN, No. P30048; SEQ ID NO:22), Ran-specific GTPase-activating protein (spot 5024; Swissprot: RANG_HUMAN, No. P43487; SEQ ID NO:23) and high mobility group protein B1 (spot 5011; Swissprot: HMGB1_HUMAN, No. P09429; SEQ ID NO:24); reactivities against tubulin beta-chain and against polymerase I and transcript release factor in spot 4672. Among these reactivities, those directed against peroxiredoxin-2, tubulin beta-chain and polymerase I and transcript release factor are specifically present in the IgGs of the patient pools and not in the IgGs of the healthy individual pool. The reactivities against thioredoxin-dependent peroxide reductase mitochondrial precursor, Ran-specific GTPase-activating protein and high mobility group protein B1 were identified in the pools of patients and of healthy individuals, but at a significantly higher level in the patients.

Furthermore, it was possible to refine the results given in example 1. The inventors were thus able to show that anti-cytoplasmic actin 2 antibodies are present in the pools of patients and of healthy individuals, but at significantly higher levels in the patients. They were also able to show the existence of reactivity against galectin-1 and zyxin in the IgGs of the patient pools, and not in the IgGs of the pool of healthy individuals.

### REFERENCES

Bordron et al, 1998, Arthritis and rheumatism, 41(10):1738-47.
Chizzolini et al, 2002, Arthritis and rheumatism, 46(6):1602-13.
Dorfmuller et al, 2003, Eur Respir J, 22(2):358-63.
Garcia de la Pena-Lefebvre et al, 2004, Clin Immunol, 111(3):241-51.
Hachulla et al, 2005, Arthritis and rheumatism, 52(12):3792-800.
Henault et al, 2006, Arthritis and rheumatism, 54(3):963-73.

Moroi et al, 1980, Proceedings of the National Academy of Sciences of the United States of America, 77(3):1627-31.
Mouthon et al, 2005, Eur Respir J, 26(6):986-8
Nicolls M R et al, 2005, Eur Respir J, 26(6):1110-8.
Rabilloud et al, 1990, Electrophoresis, 11(10):785-94.
Rubin, 1997, N Engl J Med, 336(2):111-7.
Servettaz et al, Clinical immunology, 120(2):212-9.
Tamby et al, 2005, Thorax 60(9):765-72
Tamby et al, 2006, Eur Respir J, 28(4):799-807.
Tamby et al, 2007, Annals of the New York Academy of Sciences, 1109:221-8.
Weksler et al, 2005 Faseb J, 19(13):1872-4.

1. An in vitro method for detecting systemic scleroderma and/or pulmonary arterial hypertension in an individual, or a risk of developing systemic scleroderma or pulmonary arterial hypertension, which comprises determining the presence and/or the amount of at least one antibody selected from the group consisting of the following antibodies: anti-serum albumin precursor, anti-zyxin, anti-galectin-1, anti-ubiquitin carboxyl-terminal hydrolase isozyme L1, anti-FAM10A4 protein, anti-Far-upstream element-binding protein 2, anti-cytoplasmic actin 2, anti-γ-enolase, anti-protein disulfide-isomerase A3 precursor, anti-desmin, anti-peripherin, anti-heterogeneous nuclear ribonucleoprotein H, anti-stress-induced phosphoprotein 1, anti-reticulocalbin-1, anti-peroxiredoxin-2, anti-thioredoxin-dependent peroxide reductase mitochondrial precursor, anti-Ran-specific GTPase-activating protein, anti-high mobility group protein B1, anti-tubulin beta-chain and anti-polymerase I and transcript release factor,
in a biological sample originating from a patient, the presence of said at least one antibody, or the presence of said at least one antibody in an amount greater than a control value, being an indicator of systemic scleroderma and/or of pulmonary arterial hypertension, or of a risk of developing systemic scleroderma and/or pulmonary arterial hypertension.

2. The method as claimed in claim 1, in which the biological sample is a blood or serum sample.

3. The method as claimed in claim 1, in which the presence of said at least one antibody in the biological sample is compared with a control value, the presence of said at least one antibody in an amount greater than the control value being an indicator of systemic scleroderma and/or of pulmonary arterial hypertension, or of a risk of developing systemic scleroderma and/or pulmonary arterial hypertension.

4. The method as claimed in claim 1, in which the amount of said at least one antibody is determined by means of an immunoassay.

5. The method as claimed in claim 4, in which the immunoassay is an ELISA assay.

6. The method as claimed in claim 1, in which the patient is a human being.

7. The method as claimed in claim 1, in which the patient suffers from systemic scleroderma, with or without associated pulmonary arterial hypertension.

8. The method as claimed in claim 1, in which the patient suffers from idiopathic pulmonary arterial hypertension.

9. The method as claimed in claim 1, in which the pulmonary arterial hypertension is associated with portal hypertension, with congenital heart disease, or with a human immunodeficiency virus (HIV) infection, or is post-embolic pulmonary hypertension.

10. The method as claimed in claim 1, in which the patient is an individual predisposed to developing systemic scleroderma and/or pulmonary arterial hypertension.

11. The method as claimed in claim 10, in which the individual is carrying one or more mutation(s) in the gene encoding BMPRII, endoglin or ALK1.

12. An in vitro method for the prognosis or the monitoring of systemic scleroderma and/or pulmonary arterial hypertension, which comprises determining the presence and/or the amount of at least one antibody selected from the group consisting of the following antibodies: anti-serum albumin precursor, anti-zyxin, anti-galectin-1, anti-ubiquitin carboxyl-terminal hydrolase isozyme L1, anti-FAM10A4 protein, anti-Far-upstream element-binding protein 2, anti-cytoplasmic actin 2, anti-γ-enolase, anti-protein disulfide-isomerase A3 precursor, anti-desmin, anti-peripherin, anti-heterogeneous nuclear ribonucleoprotein H, anti-stress-induced phosphoprotein 1, anti-reticulocalbin-1, anti-peroxiredoxin-2, anti-thioredoxin-dependent peroxide reductase mitochondrial precursor, anti-Ran-specific GTPase-activating protein, anti-high mobility group protein B1, anti-tubulin beta-chain and anti-polymerase I and transcript release factor,
in a biological sample originating from a patient, at various times, an increase in the amount of said at least one antibody over time being indicative of a worsening of the systemic scleroderma and/or of the pulmonary arterial hypertension.

13. An in vitro method for evaluating the efficacy of a treatment for systemic scleroderma and/or pulmonary arterial hypertension, which comprises determining the presence and/or the amount of at least one antibody selected from the group consisting of the following antibodies: anti-serum albumin precursor, anti-zyxin, anti-galectin-1, anti-ubiquitin carboxyl-terminal hydrolase isozyme L1, anti-FAM10A4 protein, anti-Far-upstream element-binding protein 2, anti-cytoplasmic actin 2, anti-γ-enolase, anti-protein disulfide-isomerase A3 precursor, anti-desmin, anti-peripherin, anti-heterogeneous nuclear ribonucleoprotein H, anti-stress-induced phosphoprotein 1, anti-reticulocalbin-1, anti-peroxiredoxin-2, anti-thioredoxin-dependent peroxide reductase mitochondrial precursor, anti-Ran-specific GTPase-activating protein, anti-high mobility group protein B1, anti-tubulin beta-chain and anti-polymerase I and transcript release factor,
in a biological sample originating from a patient, at various times before, during or after the treatment, a decrease in the amount of said at least one antibody over time being indicative of an improvement in the systemic scleroderma and/or in the pulmonary arterial hypertension.

14. The method as claimed in claim 1, said at least one antibody comprising an anti-galectin-1 antibody.

15. The method as claimed in claim 1, said at least one antibody comprising an anti-stress-induced phosphoprotein 1 antibody.
